(19) United States
(12) Patent Application Publication
(10) Pub. No.: US 2010/0168833 A1
(43) Pub. Date: Jul. 1, 2010

(54) Stent With Reduced Profile, Delivery System, and Method of Manufacture

(75) Inventor: Junghwa Jenn Cho, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic VAscular, Inc., Santa Rosa, CA (US)

(21) Appl. No.: 11/909,378

(22) PCT Filed: Mar. 7, 2006
(86) PCT No.: PCT/US06/08149

§ 371 Date: Mar. 2, 2010

Related U.S. Application Data
(60) Provisional application No. 60/665,584, filed on Mar. 24, 2005.

(51) Int. Cl. A61F 2/84; A61F 2/92; B29C 53/08

(52) U.S. Cl. 623/1.11; 623/1.15; 156/218

(57) ABSTRACT

An intraluminal stent, an intraluminal stent delivery system, and a method of manufacturing a stent. The stent (40) includes a stent body having a plurality of struts (46). The struts (46) are positioned to minimize overlap one to another when the stent is rolled at an angle. The rolled stent is in a compressed configuration. The system further includes a catheter and the stent disposed on a portion of the catheter. The manufacturing method includes providing a stent body including a plurality of struts, and helically rolling the stent at an angle to minimize overlap of the struts one to another.

### TECHNICAL FIELD OF THE INVENTION

The present invention relates generally to the field of implantable medical devices. More particularly, the invention relates to a stent with a reduced profile, delivery system, and a method of manufacture.

### BACKGROUND OF THE INVENTION

Balloon angioplasty is a medical procedure to widen obstructed blood vessels narrowed by plaque deposits. The procedure may be used in coronary or peripheral arteries. In an angioplasty procedure, a catheter having a special inflatable balloon on its distal end is navigated through the patient's arteries and is advanced through the artery to be treated to position the balloon within the narrowed region (stenosis). The region of the stenosis is expanded by inflating the balloon under pressure to forcibly widen the artery. After the artery has been widened, the balloon is deflated and the catheter is removed from the patient.

A significant difficulty associated with balloon angioplasty is that in a considerable number of cases the artery may again become obstructed in the same region where the balloon angioplasty had been performed. The repeat obstruction may be immediate (abrupt reclosure), which is usually caused by an intimal flap or a segment of plaque or plaque-laden tissue that loosens or breaks free as a result of the damage done to the arterial wall during the balloon angioplasty. Such abrupt reclosure may block the artery requiring emergency surgery which, if not performed immediately, may result in a myocardial infarction and, possibly, death. This risk also necessitates the presence of a surgical team ready to perform such emergency surgery when performing balloon angioplasty procedures. More commonly, a restenosis may occur at a later time, for example, two or more months after the angioplasty for reasons not fully understood and which may require repeat balloon angioplasty or bypass surgery. When such longer term restenosis occurs, it usually is more similar to the original stenosis, that is, it is in the form of cell proliferation and renewed plaque deposition in and on the arterial wall.

To reduce the incidence of re-obstruction and restenosis, several strategies have been developed. Implantable devices, such as stents, have been used to reduce the rate of angioplasty related re-obstruction and restenosis by about half. The use of such intraluminal devices has greatly improved the prognosis of these patients. The stent is placed inside the blood vessel after the angioplasty has been performed. A catheter typically is used to deliver the stent to the arterial site to be treated. The stent may further include one or more therapeutic substance(s) impregnated or coated thereon to limit re-obstruction and/or restenosis.

Numerous stent designs are known in the art. One consideration in the design of the stent is profile size (i.e., cross-sectional diameter). It is often desirable to provide a small profile size as advancement of a device within the vasculature oftentimes includes navigating many sharp twists, turns, and narrow spaces. Relatively large devices may be more difficult to maneuver through a sometimes tortuous vasculature. Devices with smaller profiles may be less prone to contact the vascular walls during advancement and impart damage to the delicate endothelium. As such, it would be desirable to provide a stent with a relatively small profile size.

Accordingly, it would be desirable to provide a stent with a reduced profile, delivery system, and a method of manufacture that would overcome the aforementioned and other limitations.

### SUMMARY OF THE INVENTION

A first aspect according to the invention provides an intraluminal stent. The stent includes a stent body having a plurality of struts. The struts are positioned to minimize overlap one to another when the stent is rolled at an angle. The rolled stent is in a compressed configuration.

A second aspect according to the invention provides intraluminal stent delivery system. The system further includes a catheter and a stent disposed on a portion of the catheter. The stent includes a stent body having a plurality of struts. The struts are positioned to minimize overlap one to another when the stent is rolled at an angle. The rolled stent is in a compressed configuration.

A third aspect according to the invention provides a method of manufacturing a stent. The method includes providing a stent body including a plurality of struts, and rolling the stent at an angle to minimize overlap of the struts one to another. The rolled stent is in a compressed configuration.

The foregoing and other features and advantages of the invention will become further apparent from the following description of the presently preferred embodiments, read in conjunction with the accompanying drawings. The drawings have not been drawn to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of an intraluminal stent delivery system including a compressed stent mounted on a balloon, in accordance with one embodiment of the present invention;

FIG. 2 is a perspective view the stent of FIG. 1, the stent shown in a deployed configuration in accordance with one embodiment of the present invention;

FIG. 3 is a detailed view of a ratchet of the stent shown in FIG. 2;

FIG. 4 is a perspective view of a finished form of the stent shown in FIG. 2; and

FIG. 5 is a perspective view the stent of FIG. 2, the stent shown in a compressed configuration in accordance with one embodiment of the present invention.

### DESCRIPTION OF THE PRESENTLY PREFERRED EMBODIMENTS

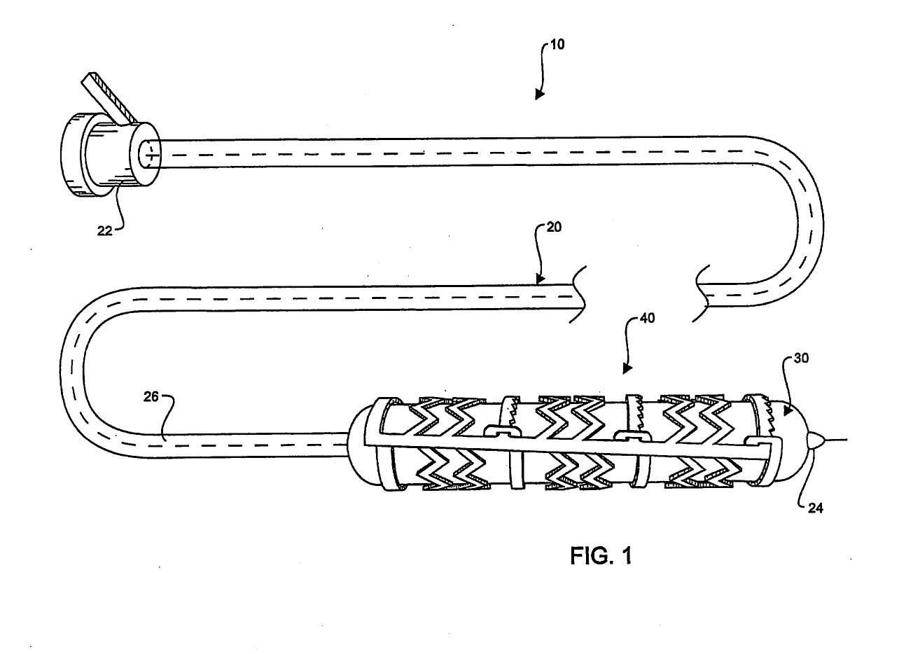

Referring to the drawings, which are not necessarily drawn to scale and wherein like reference numerals refer to like elements, FIG. 1 is a perspective view of an intraluminal stent delivery system in accordance with one embodiment of the present invention and shown generally by numeral 10. System 10 may include a catheter 20, a balloon 30 operably attached to the catheter 20, and a stent 40 disposed on the balloon 30.

Stent 40 is shown in a compressed configuration in FIG. 1 and typically remains as such on the balloon 30 during advancement through the vasculature. The compressed stent 40 includes a relatively small profile (i.e., cross-sectional size) to minimize contact with surfaces, such as a vessel wall. Once the stent 40 is properly positioned within the vasculature, the balloon 30 and stent 40 are expanded together. Balloon 30 may then be deflated and retracted thereby allowing the stent 40 to remain in a deployed configuration. The advancement, positioning, and deployment of stents and like devices are well known in the art.

In addition, those skilled in the art will recognize that numerous devices and methodologies may be adapted for deploying the stent in accordance with the present invention.

The terms “catheter” and “stent”, as used herein, may include any number of intravascular and/or implantable prosthetic devices (e.g., a stent-graft); the examples provided herein are not intended to represent the entire myriad of devices that may be adapted for use with the present invention. Although the devices described herein are primarily done so in the context of deployment within a blood vessel, it should be appreciated that intravascular and/or implantable prosthetic devices in accordance with the present invention may be deployed in other vessels, such as a bile duct, intestinal tract, esophagus, airway, etc.

Catheter 20 may comprise an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. Catheter 20 may be secured at its proximal end to a suitable Luer fitting 22, and may include a distal rounded end 24 to reduce harmful contact with a vessel. Catheter 20 may be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 20 may include an aperture formed at the distal rounded end 24 allowing advancement over a guidewire 26.

Balloon 30 may be any variety of balloons or other devices capable of expanding the stent 40 (e.g., by providing outward radial forces). Balloon 30 may be manufactured from any sufficiently elastic material such as polyethylene, polyethylene terephthalate (PET), nylon, or the like. Those skilled in the art will recognize that the stent 40 may be expanded using a variety of means and that the present invention is not limited strictly to balloon expansion.

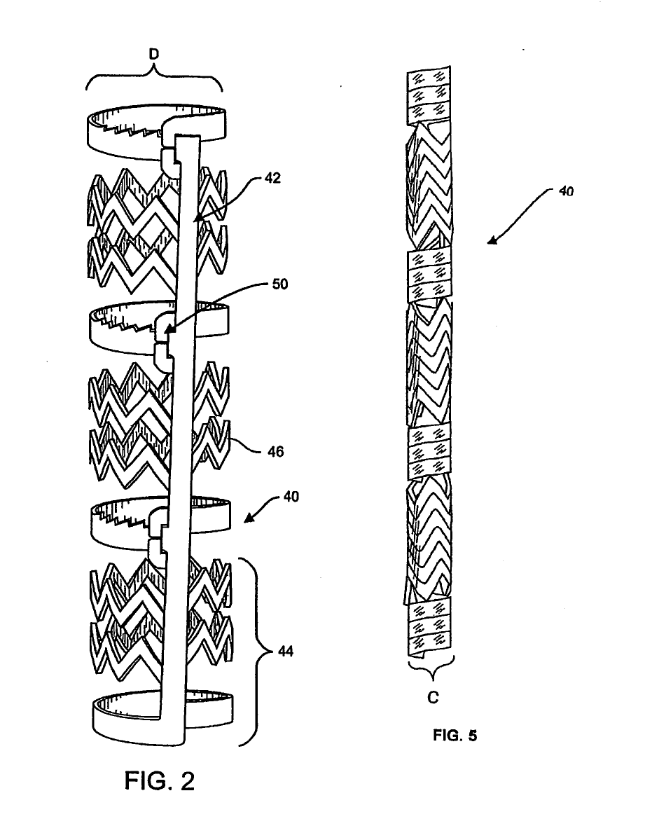

FIG. 2 is a detailed view of the stent 40 shown in a deployed configuration. In one embodiment, the stent 40 includes a generally tubular body 42 defining a passageway extending along a longitudinal axis. Stent 40 may include a plurality of cylindrical segments 44 arranged successively along the longitudinal axis. Each of the cylindrical segments 44 may have a length along the longitudinal axis and may be comprised of at least one, and in this case two, struts 46, which in this case are generally W-shaped in a repeating zig-zag form. Struts 46 may be positioned roughly parallel one to another and may extend about the perimeter or circumference of the cylindrical segments 44.

Stent 40 may include at least one, and in this case four, ratchets 50 operably attached to the stent body 42. Ratchets 50, one of which is shown in detail in FIG. 3, include a C-shaped portion 52 including an aperture formed therein for receiving a ratchet portion 54 of the stent body 42. Ratchet portion 54 may include a plurality of teeth 56 for engaging the C-shaped portion 52. Ratchets 50 allow sliding of the stent body 42 in a direction of deployment, as shown by arrow A. Ratchets 50 further minimize recoil of the stent body 42 in a direction of compression, as shown by arrow B. Those skilled in the art will recognize that the structure of the ratchet may vary from the illustrated and described embodiment.

Stent 40 is compressed into a smaller diameter (i.e., when “loaded” on the balloon) for deployment within a vessel lumen at which point the stent 40 may be expanded to provide support to the vessel. Once properly positioned within a vessel lumen, the balloon 30 and stent 40 expand together. Cylindrical segments 44 may move radially outward from the longitudinal axis as the stent 40 expands. At least one (radiopaque) marker may be disposed on the stent 40, catheter 20, and or component thereof to allow in situ visualization and proper advancement, positioning, and deployment of the stent 40. The marker(s) may be manufactured from a number of materials used for visualization in the art including radiopaque materials platinum, gold, tungsten, metal, metal alloy, and the like. Marker(s) may be visualized by fluoroscopy, IVUS, and other methods known in the art. Those skilled in the art will recognize that numerous devices and methodologies may be utilized for deploying a stent and other intraluminal device in accordance with the present invention.

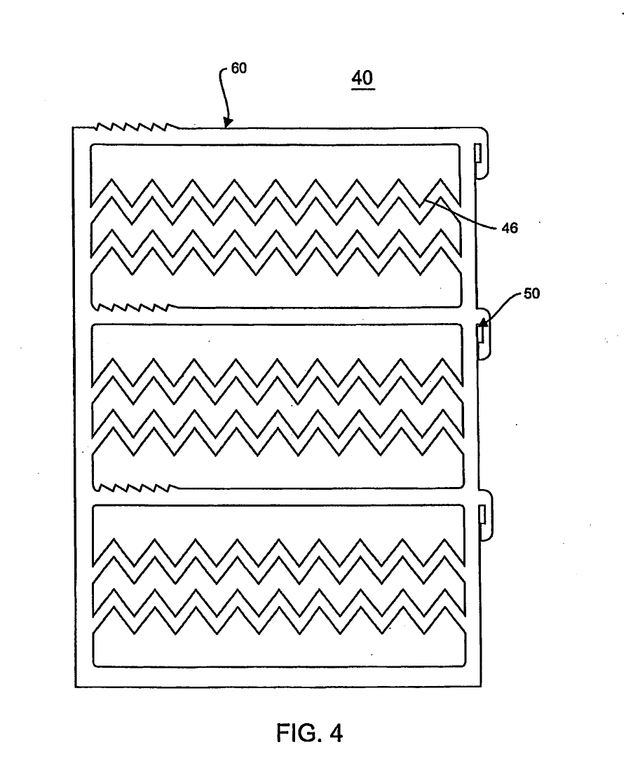

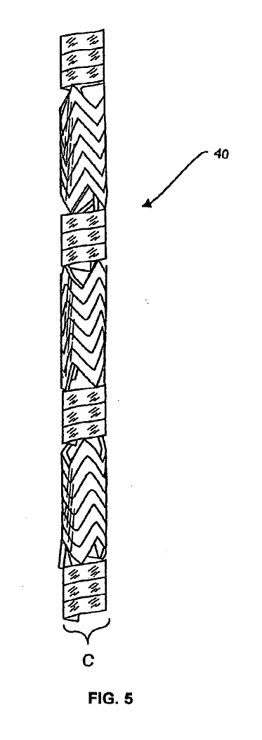

In one embodiment, the stent 40 may be manufactured from a polymer film. The film may be laser-cut as known in the art into a finished form 60 shown in FIG. 4. The finished form 60 may be rolled about three times at an angle of about four to sixteen degrees. In one embodiment, the stent 40 is rolled three times at eight degrees to compress the stent 40. The compressed stent 40 is shown in FIG. 5. A compressed stent profile size C is less than or about equal to one-third of a deployed stent profile size D, illustrated in FIG. 2. The relatively small profile size may be attributed to, at least in part, the minimal overlap of the struts 46 when rolled at an angle. Specifically, when the stent 40 is compressed, the struts 46 may be positioned more or less side-to-side with one another with little or no overlap in the radial direction. Providing the relatively small stent profile size C may reduce contact with the vascular walls during advancement and impart less damage to the delicate endothelium. Those skilled in art will recognize that the amount of times and angle of the roll may vary and are typically based on the geometry and configuration of the finished form. For example, the roll angle may be proportional to the width of the strut wherein the roll angle increases as the strut width increases. The compressed stent 40 may then be loaded onto the balloon as known in the art for subsequent deployment.

In another embodiment, the stent 40 may be manufactured from another inert, biocompatible material with high corrosion resistance. The biocompatible material should ideally be plastically deformed at low-moderate stress levels. In yet another embodiment, the stent 40 may be of the self-expanding variety and manufactured from, for example, a nickel titanium alloy and/or other alloy(s) that exhibit superlastic behavior (i.e., capable of significant distortion without plastic deformation). Other suitable materials for the stent 40 include, but are not limited to, ceramic, tantalum, stainless steel, titanium ASTM F63-83 Grade 1, niobium, high carat gold K 19-22, and MP35N. Furthermore, the stent 40 material may include any number of other polymeric biocompatible materials recognized in the art for such devices.

Stent 40 may include at least one therapeutic agent as part of one or more coatings. The coatings may be positioned on various portions of the body 42. As such, the agent(s) may be delivered to the vascular endothelium as the stent 40 biodegrades. The therapeutic agent coating may comprise one or more drugs, polymers, and the like. For example, the therapeutic agent coating solution may include a mixture of a drug and a polymer dissolved in a compatible liquid solvent as known in the art. Some exemplary drug classes that may be included are antiangiogenesis agents, antiendothelin agents, anti-inflammatory agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like.

Those skilled in the art will recognize that the nature of the drugs and polymers may vary greatly and are typically formulated to achieve a given therapeutic effect, such as limiting restenosis, thrombus formation, hyperplasia, etc. Once formulated, a therapeutic agent (mixture) comprising the coating(s) may be applied to the stent by any of numerous strategies known in the art including, but not limited to, spraying, dipping, rolling, nozzle injection, and the like. It will be recognized that the at least one therapeutic agent coating may be alternatively layered, arranged, configured on/within the stent depending on the desired effect. Before application, one or more primers may be applied to the stent to facilitate adhesion of the at least one therapeutic agent coating. Once the at least one therapeutic agent coating is/are applied, it/they may be dried (i.e., by allowing the solvent to evaporate) and, optionally, other coating(s) (e.g., a “cap” coat) added thereon. Numerous strategies of applying the primer(s), therapeutic agent coating(s), and cap coat(s) in accordance with the present invention are known in the art.

Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

While the embodiments of the invention disclosed herein are presently considered to be preferred, various changes and modifications can be made without departing from the spirit and scope of the invention. For example, the struts, number of rolls, roll angle, and stent configuration may be varied while providing a functional stent with a reduced profile.

1. An intraluminal stent comprising:
a stent body including a plurality of struts; wherein the struts are positioned to minimize overlap one to another when the stent is rolled at an angle, the rolled stent comprising a compressed configuration.

2. The stent of claim 1 wherein a compressed stent profile size is less than or about equal to one-third of a deployed stent profile size.

3. The stent of claim 1 wherein the stent comprises a polymer film.

4. The stent of claim 3 wherein the stent is laser cut.

5. The stent of claim 1 wherein the stent is rolled about three times.

6. The stent of claim 1 wherein the angle is about four to sixteen degrees.

7. The stent of claim 1 further comprising at least one ratchet operably attached to the stent body.

8. The stent of claim 7 wherein the ratchet allows sliding of the stent body in a direction of deployment and minimizes recoil in a direction of compression.

9. An intraluminal stent delivery system comprising:
a catheter; and
a stent disposed on a portion of the catheter, the stent comprises a stent body including a plurality of struts; wherein the struts are positioned to minimize overlap one to another when the stent is rolled at an angle, the rolled stent comprising a compressed configuration.

10. The system of claim 9 wherein a compressed stent profile size is less than or about equal to one-third of a deployed stent profile size.

11. The system of claim 9 wherein the stent comprises a polymer film.

12. The system of claim 11 wherein the stent is laser cut.

13. The system of claim 9 wherein the stent is rolled about three times.

14. The system of claim 9 wherein the angle is about four to sixteen degrees.

15. The system of claim 9 further comprising at least one ratchet operably attached to the stent body.

16. The system of claim 15 wherein the ratchet allows sliding of the stent body in a direction of deployment and minimizes recoil in a direction of compression.

17. A method of manufacturing a stent, the method comprising:
providing a stent body including a plurality of struts; and
rolling the stent at an angle to minimize overlap of the struts one to another, the rolled stent comprising a compressed configuration.

18. The method of claim 17 wherein a compressed stent profile size is less than or about equal to one-third of a deployed stent profile size.

19. The method of claim 17 wherein the stent comprises a polymer film.

20. The method of claim 19 wherein the stent is laser cut.

21. The method of claim 17 wherein the stent is rolled about three times.

22. The method of claim 17 wherein the angle is about four to sixteen degrees.
